(19) United States
(12) Patent Application Publication
(10) Pub. No.: US 2016/0220480 A1
(43) Pub. Date: Aug. 4, 2016

(54) ORAL DOSAGE FILM EXHIBITING ENHANCED MUCOSAL PENETRATION

(71) Applicant: IntelGenx Corp., Saint-Laurent (CA)

(72) Inventors: Mobarik Bilal, Montreal (CA); Nadine Paiement, Saint-Laurent (CA)

(73) Assignee: IntelGenx Corp., Saint-Laurent (CA)

(21) Appl. No.: 14/612,433

(22) Filed: Feb. 3, 2015

(51) Int. Cl. A61K 9/00; A61K 47/08; A61K 47/10; A61K 47/32; A61K 47/34; A61K 47/38; A61J 3/00; A61K 47/12

(52) U.S. Cl. CPC A61K 9/006; A61J 3/00; A61K 47/08; A61K 47/12; A61K 47/32; A61K 47/34; A61K 47/38; A61K 47/10

(57) ABSTRACT

A film oral dosage form useful for buccal or sublingual administration of an active agent(s), which exhibits excellent bioabsorption and rate of mucosal penetration of the active agent(s) without requiring a conventional penetration enhancer, includes at least one film forming polymer, at least one plasticizer, at least one active agent, and a residual organic solvent in an amount of from 0.5% to 8% of the weight of the film.

### CROSS-REFERENCE TO RELATED APPLICATIONS

Not Applicable.

### FIELD OF THE DISCLOSURE

This disclosure relates to oral dosage forms and more particularly to film oral dosage forms that exhibit enhanced mucosal penetration of at least one pharmaceutically active agent.

### BACKGROUND OF THE DISCLOSURE

Oral films are known to have a great potential for buccal and sublingual absorption of active agents and have a large advantage for onset of action by bypassing the liver and potential first pass metabolization and also bypassing the gastrointestinal tract (degradation due to pH and digestive enzymes. Although buccal and sublingual absorption have potential advantages, the barriers to penetrate the mucosal membrane and enter the systemic circulation to generate an efficacious level of active agents in the blood in a short period of time are numerous.

Multiple parameters impact on the amount of drug that penetrates the mucosal membrane. The oral dosage product formulation, the residence time, the surface area on the mucosa, the presence of penetration enhancer, the pH of the micro environment, and the size of the active molecule, all have an impact on the final amount of the drug(s) absorbed.

### SUMMARY OF THE DISCLOSURE

It is usually expected that surfactant and penetration enhancers have a major role in the rate and extent of absorption of a drug. Surprisingly, it was observed during development activities that the best rate and extent of drug absorption was obtained from a formulation containing neither of those, but rather a very limited quantity of solvent.

Producing an oral film involves the preparation of a wet blend into which all ingredients are dissolved, suspended or both dissolved and suspended, including a film forming polymer, a plasticizer, at least one active material and any other required ingredients. Then, the wet blend is cast into at least one thin layer and the selected solvent or solvent system is removed. A controlled and minor quantity of the solvent or solvent system remains in the film after drying, termed residual solvent(s). Active agent penetration tests using multiple distinct films showed a better rate and extent of drug penetration using the films containing no surfactant or penetration enhancer but rather residual solvent(s), as demonstrated using Franz cells set up with ex-vivo porcine and/or artificial mucosa. Surprisingly, the same film produced using water as the unique solvent, and containing water as the only residual solvent, resulted in a lower amount of active agent(s) penetrating the mucosa.

The film oral dosage forms of this disclosure includes at least one film forming polymer, at least one plasticizer, at least one active agent, and one or several residual solvent(s) representing in total between 0.5% to 8% of the weight of the film.

In certain aspects of this disclosure, the solvent or solvent system is selected from organic solvents such as alcohols, carboxylic acids, ketones, ethers, esters and/or their derivatives or from aqueous solvent or a combination of aqueous and organic solvents.

These and other features, advantages and objects of the various embodiments will be better understood with reference to the following specification and claims.

### DETAILED DESCRIPTION

The film oral dosage forms of this disclosure are useful for buccal and sublingual administration of active agents. Sublingual administration refers to the absorption of an active agent through the mucosa under the tongue, and buccal administration refers to the absorption of an active agent through the mucosa located between the gums and the cheeks. The active agent(s) can be any pharmaceutically active agent or nutraceutically active agent that is capable of being absorbed through the sublingual or buccal mucosa. Buccal and sublingual oral dosage forms are particularly useful for administering active agents that are susceptible to degradation in the gastrointestinal tract, avoiding the first pass effect of drug metabolism by the liver, or when rapid absorption of the active agent is desired.

Pharmaceutically active agents refer to any substance used in a finished pharmaceutical product and that is intended to provide pharmacological activity or otherwise have a direct effect on the diagnosis, cure, mitigation, treatment or prevention of disease, or have a direct effect in restoring, correcting or modifying physiological functions in human beings or other animals.

Nutraceutically active agents refer to dietary vitamins and minerals, and to chemicals extracted from herbs.

Pharmaceutically active agents that can be incorporated into the film oral dosage forms of this disclosure include analgesics, anti-inflammatories, antipyretics, antibiotics, laxatives, antihistamines, antiasthematics, diuretics, antiflatulents, antimigraine agents, antispasomodics, sedatives, antihyperactives, antihypertensives, tranquilizers, decongestants, beta blockers, peptides, proteins, or oligonucleotides. Film oral dosage forms for buccal and sublingual administration of an active agent are especially beneficial for treating indications where a fast onset of action is required or greatly desired, such as acute repetitive seizures, febrile seizures, acute pain, cluster headaches, agitation, acute post-operative pain, post-operative nausea and vomiting, vertigo, motion sickness, anaphylaxis, angina, and breakthrough cancer pain.

Analgesics include opiates and opiate derivatives, such as oxycodone, ibuprofen, aspirin and acetaminophen. Anti-inflammatory agents include hydroxychloroquine sulfate, fluticasone, amcinonide, methylprednisolone, budesonide, anakinra, diflorasone diacetate, and entanercept. Antipyretics include metamizole, nabumetone, phenazone and quinine. Antibiotics include amoxicillin, ampicillin, moxifloxacin hydrochloride, clarithromycin, ceftibuten, cefuroxine axetil, cefprozil, ciprofloxacin hydrochloride, clindamycin phosphate, doxycycline hyclate, dirithromycin, erythromycin, gemifloxacin, ofloxacin, telithromycin, lomefloxacin hydrochloride, minocycline hydrochloride, fosfomycin tromethamine, penicillin, trimethoprim, ciprofloxacin hydrochloride, rifampin, isoniazid, pyrazinamide, cefditeren, cefixime, tetracycline, tubramycin, rifaximin, azithromycin, linezolid, hydrocortisone, neomycin sulfate, thonzonium bromide, cephalexin hydrochloride, cefdinir, and gatifloxacin. Laxatives include prucalopride and lubiprostone. Antihistamines include acrivastine, azelastine, bilastine, brompheniramine, buclizine, bromodiphenhydramine, carbinoxamine, cetirizine, chlorpromazine, cyclizine, chlorpheniramine, chlorodiphenhydramine, clemastine, cyproheptadine, desloratadine, dextrobrompheniramine, dexchlorpheniramine, dimenhydrinate, doxylamine, ebastine, embramine, fexofenadine, hydroxyzine, levocetirizine, loratadine, meclozine, mirtazapine, olopatadine, orphenadrine, phenindamine, pheniramine, phenyltoloxamine, promethazine, pyrilamine, quetiapine, rutatadine, tripelennamine, and triprolidine. Antiasthmatics include albuterol sulfate, ipratropium bromide, salmeterol xinafoate, zafirlukast, flunisolide, metaproterenol sulfate, terbutaline sulfate, formoterol, cromolyn sodium, levalbuterol hydrochloride, zileuton, fluticasone propionate, triamcinolone acetonide, dimethylxanthine, and beclomethasone. Diuretics include spironolactone, hydrochlorothiazide, sprirolactone, butmetanide, torsemide, chlorotiazide, furosemide, and hydrochlorothiazide. Antiflatulents include simethicone, enzyme-based dietary supplements, and herbal inhibitors. Antimigraine agents include sumatriptan, rizatriptan, naratriptan, zolmitriptan, eletriptan, almetriptan, frovatriptan and avitriptan. Antispasmodics include dicyclomine, hyoscyamine, mebeverine, papaverine, cyclobenzaprime, carisoprodol, orphenadrine, tizanidine, metaxalone, methocarbamol, chlorzoxazone, baclofen, dantrolene, and baclofen. Sedatives include barbiturates, such as amobarbital, pentobarbital, secobarbital, and phenobarbital; benzodiazepines, such as clonazepam, diazepam, estazolam, flunitrazepam, lorazepam, midazolam, nitrazepam, oxazepam, trizolam, temazepam, chlordiazepoxide, and alprazolam; nonbenzodiazepines, such as eszopicione, zaleplon, zolpidem and zopiclone; suvorexant; antihistamines, such as diphenhydramine, dimenhydrinate, doxylamine, mirtazapine, and promethazine; metaqualones and methaqualone analogues, such as afloqualone, cloroqualone, diproqualone, methaqualone, methylmethaqualone, mebroqualone, mecloqualone, and nitromethaqualone; 2-methyl-2-butanol; chloral hydrate; etizolam; trazodone; and glutethimide. Antihyperactives include amphetamine, dextroamphedamine, methylphenidate, dexmethylphenidate, guanfacine, atomoxetine, and lisdexamfetamine. Antihypertensives include diuretics, such as bumetanide, ethacrynic acid, furosemide, torsemide, epitizide, hydrochlorothiazide, chlorothiasize, bendroflumethizide, idapamide, chlorthalidone, metolazone, amiloride, triamterene, and spironolactone; beta-blockers, such as atenolol, metroplol, nadolol, nebivolol, oxprenolol, pindolol, propranolol, and timolol; alpha blockers, such as doxazosin, phentolamine, indoramin, phenoxybenzamine, prazosin, terazosin, and tolazoline; mixed alpha beta blockers, such as bucindolol, carvedilol, and labelalol; benzodiazepines, such as those listed for the sedatives; calcium channel blockers, such as amlodipine, cilnidipine, felodipine, isradipine, lercanidipene, levamlodipine, nicardipine, nifedipine, nimodipine, nitrendipine, diltiazem and verapamil; rennin inhibitors, such as aliskiren; ACE inhibitors, such as captopril, enalapril, fosinopril, lisinopril, perindopril, quinapril, ramipril, trandolapril, and benazepril; angiotensin II receptor antagonists, such as candesartan, eprosartan, irbesartan, losatan, olmesartan, telmisartan, and valsartan; aldostrone receptor antagonists, such as eplerenone and spironolactone; vasodilators, such as sodium nitroprusside; alpha-2 adrenergic receptor agonists, such as clonidine, guanabenz, guanfacine, methyldopa and moxonidine; and endothelin receptor blockers, such as bosentan. Tranquilizers include anxiolytic agents, such as benzodiazepines, serotanergic, antipressants, mebicar, afobazole, selank, bromantane, emoxypine, azapirones, barbiturates, hydroxyzine, pregabalin, validol, and beta-blockers; antipsychotics, such as benperidol, brompridol, droperidol, haloperidol, moperone, pipamperone, timiperone, fluspirilene, penfluridol, pimazide, acepromazine, chlorpromazine, cyamemazine, dixyrazine, fluphenazene, levomepromazine, mesoridazine, perazine, pericyazine, pipotiazine, prochlorperazine, promazine, promethazine, prothipendyl, thioproperazine, thioridazine, trifluoperazine, triflupromazine, chlorprothixene, clopenthixol, flupentixol, tiotixene, zuclopenthixol, clotiapine, loxapine, prothipendyl, carpipramine, clocaprarnine, molindone, mosapromine, sulpiride, sultopride, veralipride, amisulpride, amoxapine, aripiprazole, asenapine, clozapine, blonanserin, iloperidone, lurasidone, melperone, nemonapride, olanzapine, paliperidone, perospirone, quetiapine, remoxipride, sertindole, trimipramine, ziprasidone, zotepine, alstonine, bifeprunox, bitopertin, brexpiprazole, cannabidiol, cariprazine, pimavanserin, pomaglumeted methionil, xanomeline, and zicronapine. Decongestants include ephedrine, levo-methamphetamine, naphazoline, oxymetazoline, phenylephrine, phenylpropanol-amine, propylhexadrine, synephrine, tetrahydrozoline, xylometazoline, pseudoephedrine, and tramazoline. These pharmaceutically active agents are illustrative, and other active agents can be used in the disclosed film oral dosage forms. Such active agents can be used alone or in combination in the disclosed film oral dosage forms.

Vitamins include Vitamin A (retinol, retinal, and carotenoids), Vitamin B1 (thiamine), Vitamin B2 (riboflavin), Vitamin 133 (niacin and niacinamide), Vitamin B5 (pantothenic acid), Vitamin B6 (pyridoxine, pyridoxamine, pyridoxal), Vitamin B7 (biotin), Vitamin B9 (folic acid and folinic acid), Vitamin B12 (cyanocobalamin, hydroxycobalamin, and methylcobalamin), Vitamin C (ascorbic acid), Vitamin D (cholecalciferol and ergocalciferol), Vitamin E (tocopherols, and tocotrienols), and Vitamin K (phylloquinone and menaquinones).

Minerals (dietary minerals or mineral nutrients) include potassium, sodium, calcium, magnesium, manganese, iron, cobalt, nickel, copper, zinc, selenium, and molybdenum, all of which are commercially available or can be prepared in biologically absorbable forms.

Other possible nutrients include fatty acids, amino acids, chloride, iodine, and phosphorus. Examples of herbal supplements include extracts from cassia cinnamon, cranberry, garlic, ginger, ginkgo, ginseng, green tea, hoodia, milk thistle, saw palmetto, St. John's wort and valerian.

These nutritionally active agents (nutraceuticals) are illustrative, and other active agents can be used in the disclosed film oral dosage forms. Such active agents can be used alone or in combination in the disclosed film oral dosage forms.

The film oral dosage forms of this disclosure can comprise one or more pharmaceutically active agent(s) in combination with one or more nutraceutically active agent(s) to treat certain conditions or combinations of conditions.

The amounts of active agent(s) incorporated into the film oral dosage forms can depend on a variety of factors including the condition being treated, the characteristics of the subject being treated (age, weight, gender, etc.), and the particular active agent(s) being employed in the film oral dosage forms. Techniques for determining appropriate dose levels of active agents for sublingual and/or buccal administration are well known, and are not the subject of this disclosure.

The major component(s) of the film oral dosage forms is at least one film forming polymer. Suitable film forming polymers are non-toxic, non-irritant and devoid of leachable impurities. The film oral dosage form should also exhibit sufficient peel, shear and tensile strength to facilitate manufacture, packaging, storage and administration without losing their integrity. In accordance with this disclosure, the selected film forming polymer(s) should be compatible with the selected organic solvent or solvent system included in the specific wet blend preparation, which is further cast to produce the film oral dosage form with a residual solvent(s) remaining after casting. Examples of film forming polymers that can be employed in the disclosed film oral dosage forms include hydroxypropylmethylcellulose, hydroxyethylcellulose, hydroxypropylcellulose, polyvinyl pyrrolidone, polymers of substituted vinylpyrrolidone, derivatives of polyvinylpyrrolidone, copolymers or polyvinylpyrrolidone, such as vinylpyrrolidone-vinyl acetate copolymers or copovidones, poly(l-vinylpyrrolidone-co-2-dimethylamino-ethyl methacrylate, poly(1-vinylpyrrolidone-co-styrene), poly(1-vinylpyrrolidone)-graft-(1-triacontene), poly (vinylpyrrolidone-co-methylacrylate), poly (vinylpyrrolidone-co-N,N′-dimethacrylamide), and poly (vinylpyrrolidone-co-maleate), polyethylene oxide, carboxymethyl cellulose, polyvinyl alcohol, natural gums, such as xanthan, tragacanth, guar, acacia and arabic gums, polydextrose, pullulan, polyacrylic acid, and polyacrylic polymers, such as methyl methacrylate polymers and copolymers.

Other polymers useful for incorporation into the film oral dosage forms of this disclosure include biodegradable polymers, copolymers, block polymers and combinations thereof. Among the known useful polymers or polymer are: poly(glycolic acid) (PGA), poly(lactic acid) (PLA), polydioxanones, polyoxalates, poly(.alpha-esters), polyanhydrides, polyacetates, polycaprolactones, poly(orthoesters), polyamino acids, polyaminocarbonates, polyurethanes, polycarbonates, polyamides, poly(alkyl cyanoacrylates), and mixtures and copolymers thereof. Additional useful polymers include, stereopolymers of L- and D-lactic acid, copolymers of bis(p-carboxyphenoxy) propane acid and sebacic acid, sebacic acid copolymers, copolymers of caprolactone, poly(lactic acid)/poly(glycolic acid)/polyethylene glycol copolymers, copolymers of polyurethane and (poly(lactic acid), copolymers of polyurethane and poly(lactic acid), copolymers of .alpha.-amino acids, copolymers of .alpha.-amino acids, and caprioc acid, copolymers of .alpha.-benzyl glutamate and polyethylene glycol, copolymers of succinate and poly(glycols), polyphosphazene, polyhydroxy-alkanoates and mixtures thereof. Binary and ternary systems are contemplated.

Other specific polymers that can be used in the film oral dosage forms of this disclosure include those marketed under the Medisorb and Biodel trademarks. The Medisorb materials are marketed by the DuPont Company of Wilmington, DE and are generically identified as a “lactide/glycolide co-polymer” containing propanoic acid, 2-hydroxy-polymer with hydroxyl-polymer with hydroxyacetic acid.” Such polymers include lactide-glycolide 100 L, believed to be 100% lactide, having a melting point within the range of 338°-347° F. (170°-175° C.); lactide/clycolide 100 L, believed to be 100% glycolide having a melting point within the range of 437°-455° F. (225°-235° C.); lactide/glycoli 85/15, believed to be 85% lactide and 15% glycolide, having a melting point within the range of 338°-347° F. (170°-175° C.); and lactide/glycolide 50/50, believed to be a copolymer of 50% lactide and 50% glycolide, having a melting point within the range of 338°-347° F. (170°-175° C.).

The film forming polymers typically represent 30% to 95% of the weight of the cast films prior to packaging, e.g., 40% to 95%, 50% to 95%, 60% to 90%.

The film oral dosage forms disclosed herein can be free or substantially free of surfactants and polyalcohols. The term “substantially free” of surfactants and polyalcohols means that the film oral dosage forms do not require deliberately added surfactants or polyalcohols, although unavoidable or incidental surfactants or polyalcohols can be present, if at all, as impurities in other ingredients in amounts that do not affect measurable properties relating to wettability (e.g., contact angle goniometer measurements), dissolution or disintegration rates by more than 10%, and do not adversely affect measurable stability properties. The term “free” of surfactants and polyalcohols means that incidental or unavoidable surfactants and polyalcohol impurities are present in only inconsequential amounts that affect water contact angle measurements and dissolution rate by less than 1%. In certain embodiments, the presence of surfactants and polyalcohols are each limited to less than 1000 ppm (w/w), less than 500 ppm (w/w), less than 100 ppm (w/w), less than 40 ppm (w/w), or less than 10 ppm (w/w).

The terms “surfactant” and “polyalcohol” are intended to have their ordinary meanings. Specifically, the term “surfactant” is intended to mean an amphophilic compound that lowers the surface tension of a liquid, the interfacial tension between two liquids, or the interfacial tension between a liquid and a solid. The term “polyalcohol” means a sugar alcohol, which is a hydrogenated form of a carbohydrate having a carbonyl group that has been reduced to a primary or secondary hydroxyl group. Polyalcohols are also distinguishable based on their chemical formula. Polyalcohols have the general formula H(HCHO)ₙ₊₁, whereas sugars have the general formula H(HCHO)ₙHCO. Common examples of polyalcohols or sugar alcohols that can be avoided or eliminated from the disclosed films include glycol, glycerol, erythritol, threitol, arabitol, xylitol, ribitol, mannitol, sorbitol, galactitol, fucitol, iditol, inositol, volemitol, isomalt, maltitol, lactitol, maltotritol and maltotetraitol.

Titanium dioxide can be added in amounts from about 0.05% to 5%, 0.1% to 3%, or 0.5% to 2% of the weight of the film. The titanium dioxide can act as a disintegrant in the disclosed films, as well as a texture modifier that improves mouth feel, and an opacifier or coloring agent. These amounts are effective for increasing the rate at which the film will dissolve in an aqueous medium (e.g., saliva) upon buccal and/or sublingual administration.

The residual solvent(s) is the remaining solvent contained in the finished film product. The solvent is selected based on its capacity to dissolve the film forming polymer(s) that can be used to produce a wet blend from which a film can be obtained by removal (e.g., evaporation) of most of the solvent(s). Examples of suitable organic solvents or solvent system include carboxylic acids, ketones, ethers, esters and/or their derivatives. Carboxylic acids that can be used as the organic solvent include formic acid, acetic acid, propanoic acid, butyric acid, and valeric acid. Alcohols that can be used as the organic solvent include 1-butanol, 2-butanol, ethanol, 3-methyl-1-butanol, 2-methyl-1-propanol, 1-pentanol, 1-propanol and 2-propanol. Ketones that can be used as the organic solvent include acetone, methylethyl ketone, and methylisobutyl ketone. Esters that can be used as the organic solvent include butyl acetate, ethyl acetate, ethyl formate, isobutyl acetate, isopropyl acetate, propyl acetate, and methyl acetate. Ethers that can be used as the organic solvent include tert-butylmethyl ether and ethyl ether. Combinations of these or other organic solvents can be used.

In a single layer or monolayer film, the amount of residual solvent is about 0.5% to 8% of the weight of the film. In a multiple layer film, at least one of the layers contains residual organic solvent in an amount of about 0.5% to 8% of the weight of the film layer.

In certain embodiments, the disclosed films may include a plasticizer. The term “plasticizer” refers to a component that reduces the glass-transition temperature of the film forming polymers (e.g., the water soluble polymer or water soluble polymers in the film). The plasticizer increases the flexibility, enhances elasticity, and reduces brittleness of the film. Examples of plasticizers that can be used in the disclosed film oral dosage forms include triacetin, triethyl citrate, tributyl citrate, acetyl tributyl citrate, acetyl triethyl citrate, trioctyl citrate, acetyl trioctyl citrate, trihexyl citrate, dibutyl sebacate, etc. Other plasticizers include polyalkylene oxides, glycerol, glycerol monoacetate, diacetate or triacetate, polysorbate, cetyl alcohol, sorbitol and sodium diethylsulfosuccinate. Plasticizer may be added in an amount up to 25% of the total mass of the film oral dosage form, such as 0.5% to 25%, 1% to 20%, 2% to 15% or 5% to 10%.

The amount of active agent(s) that can be incorporated in the film oral dosage forms disclosed herein is generally from 0.01% to 50% by total weight of the film, such as 1% to 40%, 2% to 30%, or 5% to 20% by total weight of the film.

Conventional film oral dosage form additives, other than surfactants and polyalcohols, can be added as needed or desired, generally in amounts conventionally employed. Examples of such additives include artificial sweeteners such as sucralose, aspartame, acesulfame potassium and monoammonium glycyrrhizinate; natural sweeteners such as sucrose and fructose; flavorants such as menthol, various fruit flavors (e.g., citrus, cherry, grape, orange, etc.) or various mint flavors (e.g., spearmint, peppermint, etc.); colorants; opacifiers (e.g., titanium dioxide); and antioxidants (e.g., butylhydroxytoluene). Other additives may also be incorporated in amounts that do not adversely affect the film properties or film stability. Specifically, any such additives must not cause undesirable softening of the film and subsequent loss of dimensional stability, degradation of the active ingredient(s), or induce undesirable aesthetics such as discoloration of the film or noticeable segregation and agglomeration of film components.

In an embodiment, a method of forming a film of the present disclosure includes combining the various ingredients in generally any order, employing a combination of water and water-miscible solvents such as alcohols (e.g., ethanol) or organic solvents alone or as a mixture. For example, the plasticizer and additives (e.g., sweetening agents, colorants, flavorants, and opacifying agents) can be dissolved or dispersed in a sufficient amount of solvent that is agitated to form a homogenous solution or suspension to which the water soluble polymer(s) is (are) added. Heat, vacuum and agitation may be applied as needed during addition of the water soluble polymer until a homogenous solution or homogenous suspension is obtained. Thereafter, the active ingredient(s) is (are) added. Eventually, the active ingredient(s) may be homogeneously dissolved or suspended within the solvent or solvent system before the addition of any other required ingredients or after a specific ingredient, depending on the properties of the wet blend preparation (e.g., viscosity) and the oral film dosage form (e.g., dimensions, air bubbles, residence time). The solution or suspension is cast or coated onto a carrier material and dried to form a film. Examples of suitable carrier materials include non-siliconized polyethylene terephthalate film, non-siliconized kraft paper, polyethylene-impregnated kraft paper and non-siliconized polyethylene film. The liquid film composition can be coated onto the carrier material using generally any conventional coating equipment, including knife-over-roll, extrusion die, reverse roll, or Meyer roll coating equipment.

Upon drying, the resulting solid film can have a thickness of generally 5 to 200 μm, such as 10 to 200 μm, 20 to 150 μm or 20 to 100 μm. The film can be cut into individual pieces having a suitable size to facilitate administration of a targeted dosage of active agent(s).

The film oral dosage forms of this disclosure can be formulated without conventional penetration enhancers used to promote buccal and/or sublingual transmucosal absorption of active agent(s), such as sulfoxides (e.g., dimethylsulfoxide), azones (e.g., laurocapran), pyrrolidones (e.g., 2-pyrrolidone), and glycols (e.g., propylene glycol).

The amount of residual solvent in the film oral dosage forms can be controlled by selecting drying times and conditions (e.g. heating temperature, ventilation, and drying sequence) that consistently provide the desired residual amounts (e.g., 0.5 to 8%, 1% to 4%, or 1% to 3% of the weight of the film), as may be determined using analytical techniques such as gas chromatography.

Once the product is in its finished form, the film dosage forms are rapidly packaged individually to prevent evaporation of the residual solvent(s) during the normal shelf life of the particular product.

The above description is considered that of the preferred embodiment(s) only. Modifications of these embodiments will occur to those skilled in the art and to those who make or use the illustrated embodiments. Therefore, it is understood that the embodiment(s) described above are merely exemplary and not intended to limit the scope of this disclosure, which is defined by the following claims as interpreted according to the principles of patent law, including the doctrine of equivalents.

1. A film oral dosage form or film layer of an oral dosage form comprising:
at least one film forming polymer;
at least one plasticizer in an amount of from 1% to 20% of the total mass of the film;
an optional surfactant in an amount that does not exceed 1000 ppm (w/w);
at least one active agent; and
at least one residual organic solvent in an amount of from 0.5% to 8% of the weight of the film or film layer, wherein the oral dosage form is packaged to prevent evaporation of the residual organic solvent.

2. The film oral dosage form of claim 1, which does not contain a surfactant.

3. The film oral dosage form of claim 1, which does not contain a mucosal penetration enhancer.

4. The film oral dosage form of claim 1, which does not contain either a surfactant or a mucosal penetration enhancer.

5. The film oral dosage form of claim 1, in which the at least one residual solvent is selected from alcohols, carboxylic acids, ketones, ethers, esters and their derivatives.

6. The film oral dosage form of claim 1, in which the film or film layer further comprises residual water.

7. A process for making a film oral dosage form or film layer for an oral dosage form, comprising:
forming a wet blend of at least one film forming polymer, at least one plasticizer in an amount of from 1% to 20% of the total mass of the film; an optional surfactant in an amount that does not exceed 1000 ppm (w/w); and at least one active agent in a liquid including at least one organic solvent;
applying at least one layer of the wet blend onto a substrate;
casting a film by removing most of the liquid, while retaining residual organic solvent in an amount of 0.5% to 8% of the weight of the film or film layer; and
packaging the oral dosage form to prevent evaporation of the residual organic solvent.

8. The process of claim 7, in which the film does not contain a surfactant.

9. The process of claim 7, in which the film does not contain a mucosa penetration enhancer.

10. The process of claim 7, in which the film does not contain either a surfactant or a mucosa penetration enhancer.

11. The process of claim 7, in which the organic solvent is selected from alcohols, carboxylic acids, ketones, ethers, esters and/or their derivatives.

12-15. (canceled)
